(19) United States
(12) Patent Application Publication
(10) Pub. No.: US 2003/0199693 A1
(43) Pub. Date: Oct. 23, 2003

(54) Theophylline and 3-isobutyl-1-methylxanthine based N-7 substituted derivatives displaying inhibitory activies on PDE-5 phosphodiesterase

(76) Inventor: Ing-Jun Chen, Kaohsiung (TW)

Correspondence Address: BUCKNAM AND ARCHER, 1077 Northern Boulevard, Roslyn, NY, 11576-1696, US

(21) Appl. No.: 10/342,650

(22) Filed: Jan. 15, 2003

Related U.S. Application Data
(63) Continuation-in-part of application No. 09/906,245, filed on Jul. 16, 2001, now abandoned.

(30) Foreign Application Priority Data
Jul. 28, 2000 (TW) 89115304

(51) Int. Cl. A61K 31/522; C07D 473/02

(52) U.S. Cl. 544/269; 514/252.16

(57) ABSTRACT

Compounds of the theophylline and 3-isobutyl-1-methylxanthine (IBMX) based on N-7 substituted derivatives, X being florine, chlorine, bromine or iodine, are provided. These compounds possess pharmacologically inhibitory activities on PDE-5 Phosphodiesterase, relaxation of corpus carvernosal smooth muscle and increase of intracarvernosal pressure (ΔICP). A process is also provided for the synthesis of some novel theophyline derivatives.

This application is a Continuation-in-Part of U.S. Ser. No. 09/906,245 filed Jul. 16, 2001.

### FIELD OF THE INVENTION

This invention relates to compounds of theophylline and 3-isobutyl-1-Methylxanthine (IBMX) based on N-7 substituted derivatives, X being F, Cl, Br or I, which upon laboratory testing on animals, have proved that they pharmacologically possess inhibitory activities on PDE-5 Phosphodiesterase, relaxation of corpus carvernosal smooth muscle and increase of intracarvernosal pressure (ΔICP).

### BACKGROUND OF THE PRIOR ART

The endothelium plays a major role in regulating vascular smooth muscle (VSM) tone through the release of a variety of vasoactive factors. Among the endothelium-derived vasodilators, nitric oxide (NO) is probably the primary mediator of endothelium-dependent relaxation in most blood vessels. Nitric oxide in numerous bioregulatory pathways has not only expanded new therapeutic avenues for NO-related compounds but has also led to an increased use of such compounds in pharmacological studies.

In recent years, nitric oxide has been shown to be an important regulator of vascular functions by controlling blood vessel tone as well as blood cell interactions with the vascular wall (S. Moncada et al., Pharmacol. Rev. vol. 43, No. 2, pp. 109-142, 1991). The action of NO (nitric oxide) as a vasodilator is mediated by the activation of vascular smooth muscle soluble guanylyn cyclase (sGC), a signal transduction enzyme which forms the second messenger of molecular cyclic GMP (William P. Arnold et al., Proc. Natl. Acad. Sci. vol. 74, No. 8, pp. 3203-3207, 1977, Charles J. Lowenstein et al., Ann. Intern. Med. vol. 102, No. 3, pp. 227-237, 1994). The activity of several cyclic GMP (guanosine 3′, 5′-cyclic monophosphate) which lead to vasorelaxation has been determined. The membrane-bound guanylyl cyclases are receptor-like enzymes which are activated by extracellular binding of natriuretic peptides. In contrast, soluble guanylyl cyclases act via their hemoglobin group which is an important intracellular receptors for nitric oxide (Paulus Wohfart et al., Br. J. Pharmcol. vol. 128, pp. 1316-1322, 1999). Moreover, the increases in cGMP with these guanylyl cyclase activators and phosphodiesterases (PDE) or cGMP breakdown inhibition which have been associated with the relaxation of vascular and tracheal smooth muscles.

The interactions between endogenous NO or NO donors and endothelium-derived hyperpolarizing factor (EDHF) or K+ channels have received a great deal of attention. (Francisco Perez-Vizcaino, et al. British J. Pharmacol. Vol. 123, pp. 847-854, 1998). K+ channels play a major role in the regulation of the resting membrane potential and modulate VSM (vascular smooth muscle) tone (Mark T. Nelson & John M. Quayle, Am. J. Physiol. vol. 268, C799-C822, 1995). The endothelium-derived hyperpolarizing factor activates the potassium channels, and the potassium flux hyperpolarizes and thus relaxes the smooth muscle cell. Recent findings suggest that activation of endothelium Kₐₜₚ channels (ATP-sensitive potassium channels) may also release endothelium-derived nitric oxide (Ethel C. Feleder & Edda Adler-Graschinsky, Eur. J. pharmacol. vol. 319, pp. 229-238,1997) or endothelium derived hyperpolarizing factor (Richard White and C. Robin Hiley, Eur. J. Pharmacol. vol. 339, pp. 157-160, 1997). Nitric oxide donors have been shown to activate Kₐₜₚ channels via a cyclic GMP-dependent mechanism, presumably involving activation of cyclic GMP-dependent protein kinase, in rat aortic smooth muscle cells (Masahiro Kubo et al., Circ. Res. vol. 74, No. 3, pp. 471-476, 1993) and rabbit mesenteric artery (Michael E. Murphy & Joseph E. Brayden, J. Physiol. vol. 486, No. 1, pp. 47-48, 1995), and by a cyclic GMP-independent mechanism in the rat mesenteric artery (Thomas Weidelt et al., J. Physiol. vol. 500, No. 3, pp. 617-630, 1997). Although most of the endothelium-dependent relaxation is due to NO (nitric oxide), hyperpolarization associated with K+ channels opening can supplement 60-80% of this response if NO synthesis is blocked (E. V. Kilpatrick & T. M. Cocks, Br. J. Pharmacol. vol. 112, pp. 557-565, 1994)

The combination activity of soluble guanylyl cyclase (sGC) stimulation and K+ channels opening in a molecule, such as found in nicorandil, although shown without phosphodiesterase (PDE) inhibition activity, is able to relax agonist-induced vasoconstriction more fully (F. Perez-Vizcaino et al., Br. J. Pharmcol. vol. 123, pp. 847-854, 1998). YC-1(3-(5′hydroxymethyl-2furyl)-1-benzyl-indazole) is a representative of a class of sGC activator with PDE (phosphodiesterase) inhibition and leads to a long-lasting cyclic GMP-mediated inhibition of vasoconstriction (Jan Gaile et al., Br. J. Pharmcol. vol 127, pp. 195-203, 1999).

### SUMMARY OF THE INVENTION

This invention covers compounds of theophylline and 3-isobutyl-1-Methylxanthine (IBMX) based on N-7 substituted derivatives, X being F, Cl, Br or I, which on laboratory testing on animals, have proved that they pharmacologically possess inhibitory activities on PDE-5 Phosphodiesterase. This invention also covers the synthetic methods of some novel theophylline derivatives.

This invention contains compounds I and II as shown in the following pages.

A compound containing the theophylline moiety of formula I,

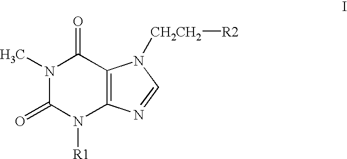

wherein

R¹ is —(CH₂)_{c}CH₃; R₂ is a member selected from the group

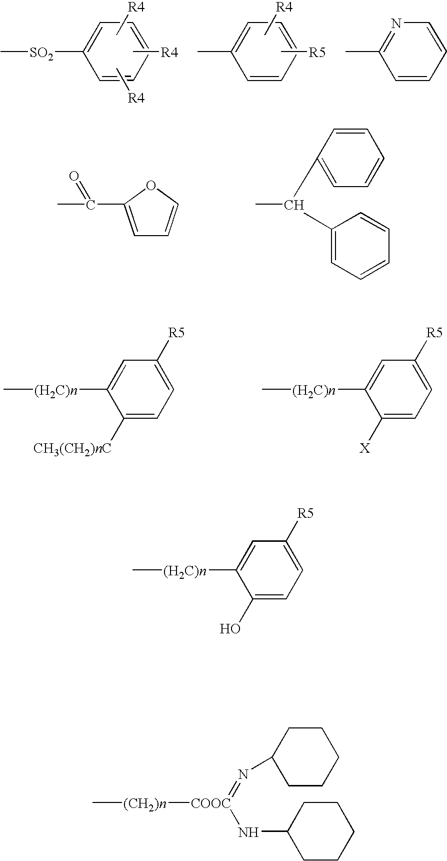

wherein

R₄ is a member selected from the group of H, —(CH₂)ₙCH₃,X,—NH₂ and —NO₂ and X designates F, Cl, Br or I,

R₅ is a member selected from the group of H,

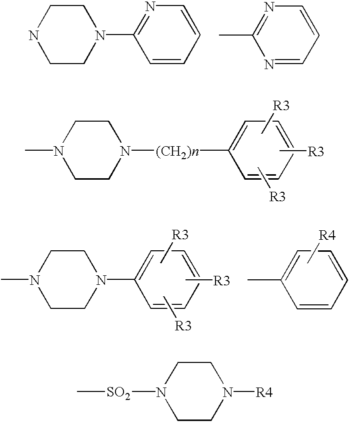

wherein

R₃ is a member selected from the group of halogen, hydroxyl group, a saturated straight chain of 1-3 carbon atoms and when one of R₃ is hydrogen, n is 0, 1, 2 or 3.

2.

A compound containing the theophylline moiecy of formula II,

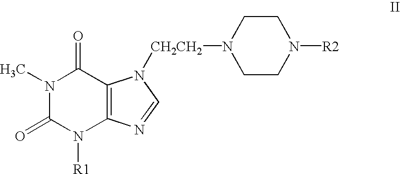

wherein

R₁ is —(CH₂)ₙCH₃; R₂ is a member selected from the group of

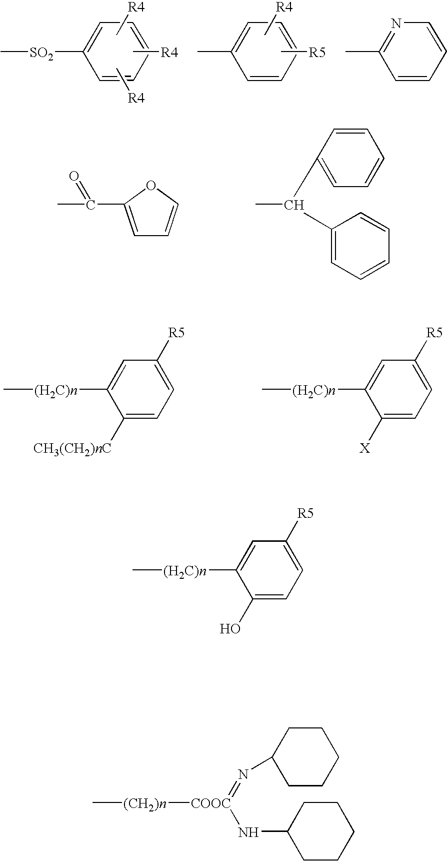

wherein

R₄ is a member selected from the group of H, —(CH₂)ₙCH₃,X, —NH₂ and —NO₂; X designated F, Cl, Br or I,

R₃ is a member selected from the group of H

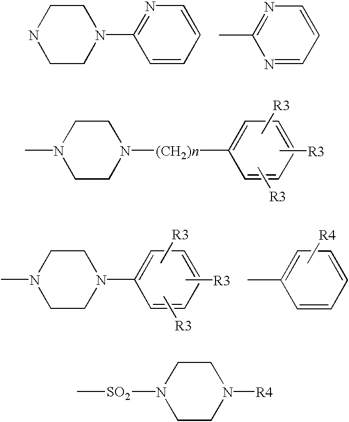

R₃ is a member selected from the group of halogen, hydroxyl group (OH), a saturated straight chain of 1-3 carbon atoms and when one of R₃ is hydrogen; n is 0, 1, 2 or 3.

Many compounds are listed with a number. Exhibits 1 and 2 show the chemical structure of these compounds. Specifically, Exhibit 1 shows the chemical structure of compounds 27, 28, 29, 30, 31, 32, 33, 34, 35, 36, 37, 38, 39, 40 and 41.

Exhibit 2 shows the chemical structure of compounds 1, 2, 3, 4, 5, 6, 7, 8, 9, 10, 11, 12, 13, 14, 15, 16, 17, 18, 19, 20, 21, 22, 23, 24, 25 and 26.

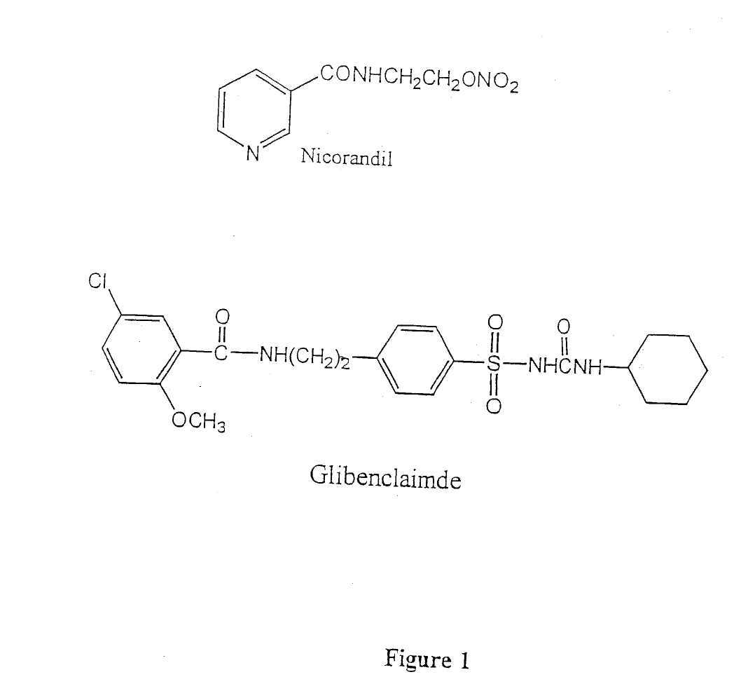

FIG. 1 illustrates the structure of Nicorandil and glibenclamide.

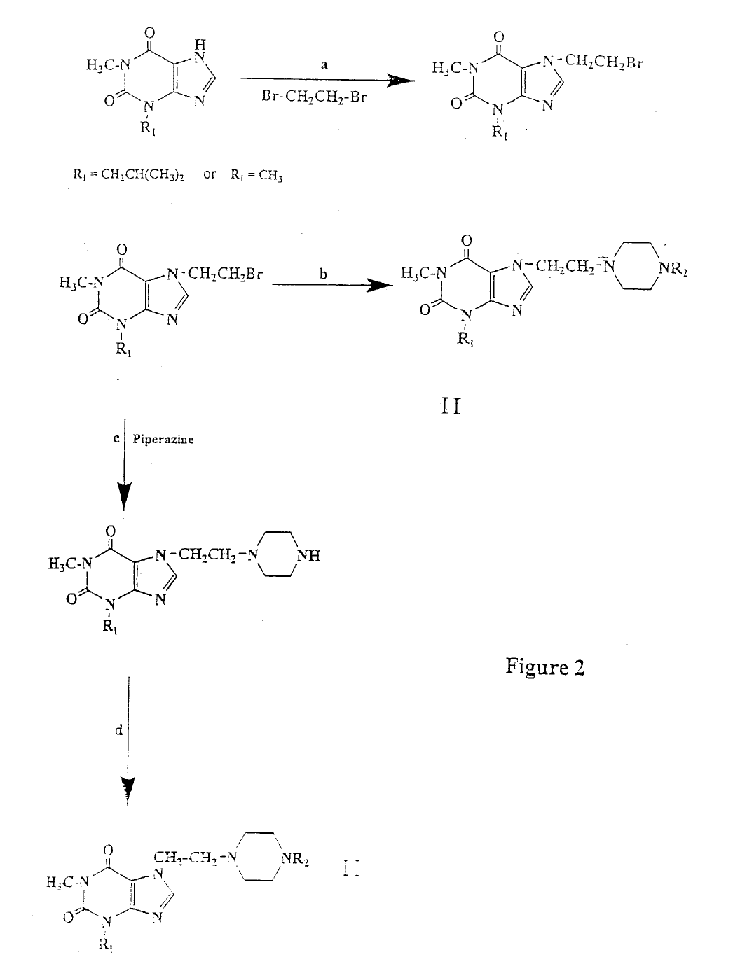

FIG. 2 illustrates the synthetic method for the compounds of formula II.

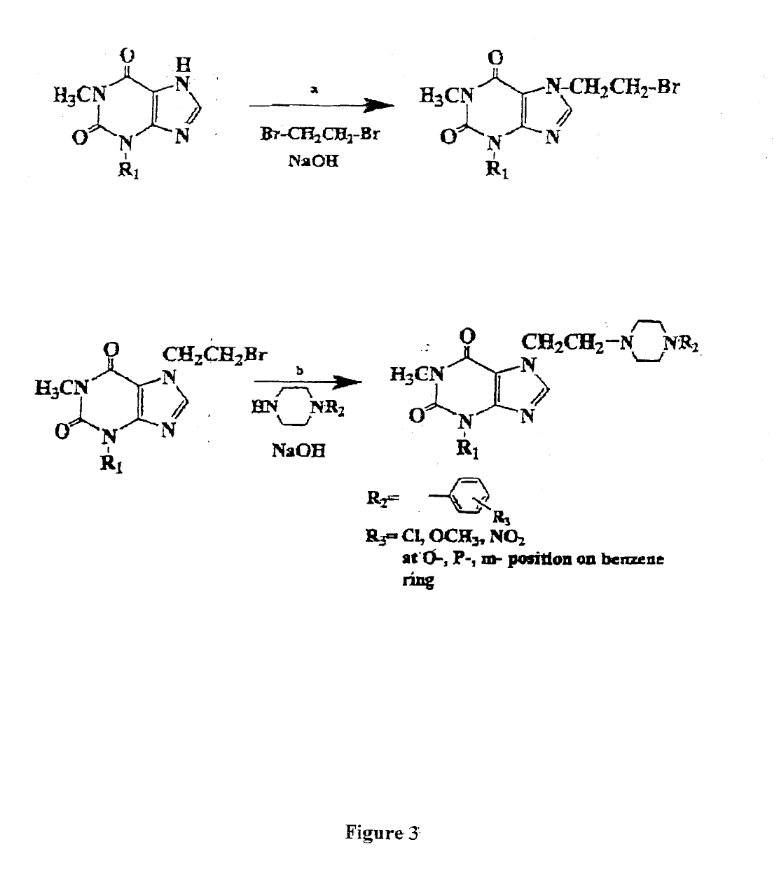

FIG. 3 illustrates the synthetic method for the compounds of formula I.

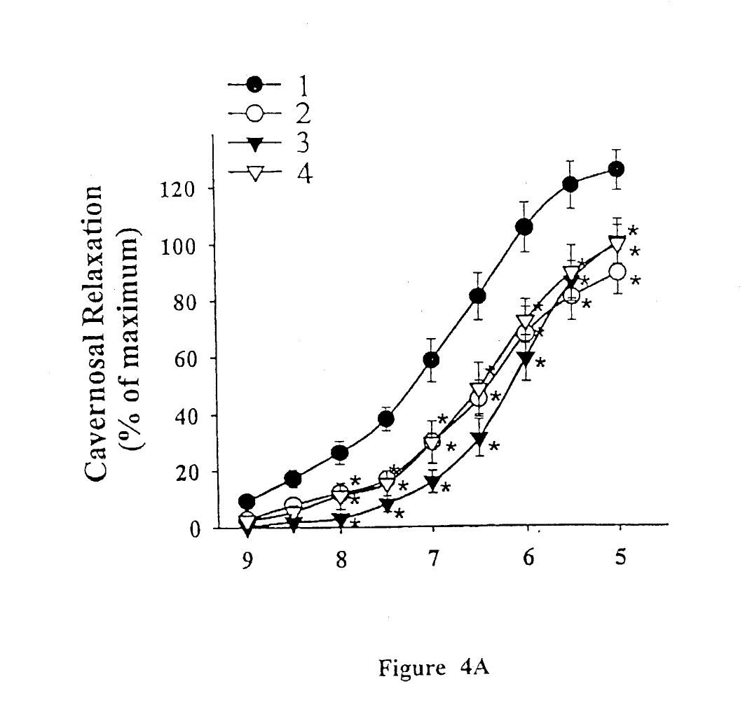

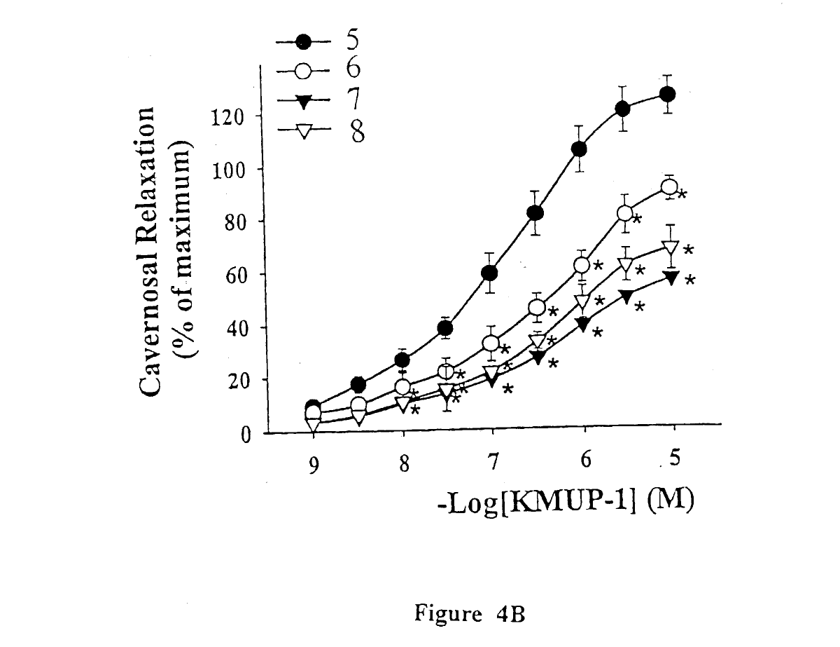

FIGS. 4A-4B illustrates the experiment, effects of compound 14 on phenylephrine retracted rabbit corpus carvernosal in the absence and presence of L-NAME, methylene blue, ODQ and potassium channel blockers. Significantly different from control, P<0.05 (two-way repeated measures NOVA followed by student Newman-Keuls test).

FIG. 4A illustrates the experiments, effects of compound 14 on phenylephrine precontracted rabbit corpus carvernosal in the absence and presence of L-NAME, methylene blue, ODQ.

1 . . . control group

2 . . . L-NAME (100 μM)

3 . . . methylene blue (10 μM)

4 . . . ODQ (1 μM)

FIG. 4B illustrates the experiments, effects of compound 14 on phenylephrine precontracted rabbit corpus carvernosal in the absence and presence of potassium channel blockers.

5 . . . control group

6 . . . Glibenclamide (1 μM)

7 . . . TEA (10 μM)

8 . . . 4-AP (100 μM)

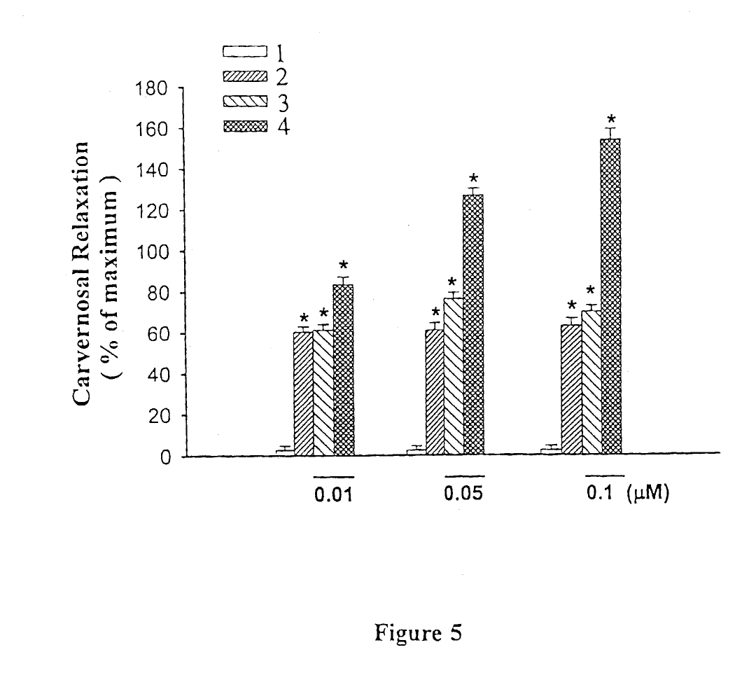

FIG. 5 illustrates the additive effects of compound 14 and IBMX (3-Isobutyl-1-Methylxanthine) X being F, Cl, Br or I, on phenylephrine precontracted rabbit carvernosal strips. Each value represents the mean*S.E., n=8. *P<0.05 as compared with the control value. (ANOVA followed by Dunnett's test). The superiority in the relaxation of corpus carvernosal of the additive effect of compound 14 and IBMX (3-Isobutyl-1-Methylxanthine) X being F, Cl, Br or I), is manifest.

1 . . . control group

2 . . . IBMX

3 . . . compound 14

4 . . . compound 14 and IBMX 3-Isobutyl-1-Methylxanthine)

X being F, Cl Br or I.

### BRIEF DESCRIPTION OF THE TABLES

The invention will now be described with reference to the accompanying Tables in which:

Table 1 Physicochemical data.

Table 2 Rabbit Corpus Carvernosal Relaxation IC_{50.}

Table 3 Rabit Corpus Carvernosal Relaxation IC₅₀ on K⁺ channels blocker.

Table 4 Show the increase of intracarvernous pressure (AICP) induced by the compounds.

TABLE 1
The physiocochemical Data of N7-substituted xantines

| | | |
|---|---|---|
| | | Compound |
| MS(Scan | | ¹H-NMR(CD Cl₃) |
| FAB÷) | | |
| | | Compound 4 |
| 444.88 | δ: | 0.94-0.98 (d, 6H, 2xCH₃), |
| | | 2.70 (t, 4H, 2xCH₂ ), |
| | | 3.04 (t, 4H, 2xCH₂), |
| | | 3.93-3.96 (d, 2H, CH₂), |
| | | 6.97-7.01 (m, 2H, 2xAr—H), |
| | | 7.69 (s, 1H, imidazole-H) |
| | | 2.24-2.38 (m, 1H, CH) |
| | | 2.85 (t, 2H, NCH₂), |
| | | 3.42 (s, 3H, NCH₃), |
| | | 4.45 (t, 2H, NCH₂), |
| | | 7.27-7.36 (m, 2H, 2xAr—H), |
| | | Compound 14 |
| 402.88 | δ: | 2.70 (t, 4H, 2xCH₂), |
| | | 3.04 (t, 4H, 2xCH₂), |
| | | 3.60 (s, 3H, NCH₃), |
| | | 6.97-7.01 (m, 2H, 2xAr—H), |
| | | 7.69 (s, 1H, imidazole-H) |
| | | 2.85 (t, 2H, NCH₂), |
| | | 3.42 (s, 3H, NCH₃), |
| | | 4.45 (t, 2H, NCH₂), |
| | | 7.27-7.36 (m, 2H, 2xAr—H) |
| | | Compound 17 |
| 398.46 | δ: | 2.75 (t, 4H, 2xCH₂), |
| | | 3.09 (t, 4H, 2xCH₂), |
| | | 3.61 (s, 3H, NCH₃), |
| | | 4.49 (t, 2H, NCH₂), |
| | | 7.72 (s. 1H, imidazole-H) |
| | | 2.89 (t, 2H, NCH₂), |
| | | 3.42 (s, 3H, NCH₃), |
| | | 3.86 (s, 3H, OCH₃), |
| | | 6.88-7.06 (m, 4H, 4xAr—H), |
| | | Compound 22 |
| 488.38 | δ: | 0.94-0.98 (d, 6H, 2xCH₃), |
| | | 2.24-2.38 (m, 1H, CH), |
| | | 2.85 (t, 2H, NCH₂), |
| | | 3.42 (s, 3H, NCH₃), |
| | | 4.45 (t, 2H, NCH₂), |
| | | 7.27-7.36 (m, 2H, 2xAr—H), |
| | | 1.98 (m, 3H, Ar—CH₃) |
| | | 2.70 (t, 4H, 2xCH₂), |
| | | 3.04 (t, 4H, 2xCH₂), |
| | | 3.93-3.96 (d, 2H, CH₂), |
| | | 6.97-7.01 (m, 2H, 2xAr—H), |
| | | 7.69 (s. 1H, imidazole-H), |
| | | Compound 25 |
| 446.38 | δ: | 1.98 (m, 3H, Ar—CH₃) |
| | | 2.85 (t, 2H, NCH₂), |
| | | 3.42 (s, 3H, NCH₃), |
| | | 4.45 (t, 2H, NCH₂), |
| | | 7.27-7.36 (m, 2H, 2xAr—H). |
| | | 2.70 (t, 4H, 2xCH₂), |
| | | 3.04 (t, 4H, 2xCH₂), |
| | | 3.60 (s, 3H, NCH₃), |
| | | 6.9-7.01 (m, 2H, 2xAr—H), |
| | | 7.69 (s, 1H, imidazole-H). |
| | | Compouud 34 |
| 392.14 | δ: | 0.94-0.98 (d, 6H, 2xCH₃), |
| | | 2.24-2.33 (m, 1H, CH) |
| | | 2.85 (t, 2H, NCH₂) |
| | | 3.93-3.96 (d, 2H, CH₂), |
| | | 4.45 (t, 2H, NCH₂), |
| | | 7.22-7.36 (m, 1H, Ar—H), |
| | | 7.98 (b, 1H, NH), |
| | | 1.4 (t, 2H, CH₂), |
| | | 2.28 (s, 2H, CH₂), |
| | | 3.42 (s, 3H, NCH₃) |
| | | 4.13 (t, 3H, CH₃) |
| | | 6.97-7.01 (m, 2H, 2xAr—H) |
| | | 7.69 (s, 1H, imidazole-H), |
| | | 8.2 (b, 2H, NH₂) |
| | | Compound 38 |
| 364.14 | δ: | 1.4 (t, 2H CH₂), |
| | | 2.28 (s, 2H, CH₂), |
| | | 3.42 (s, 3H, NCH₃), |
| | | 4.13 (t, 3H, CH₃), |
| | | 6.97-7.01 (m, 2H, 2xAr—H), |
| | | 7.69 (s, 1H, imidazole-H) |
| | | 8.2 (b, 2H, NH₂) |
| | | 2.2 (t, 2H, CH₂), |
| | | 2.85 (t, 2H, NCH₂), |
| | | 3.60 (s, 3H, NCH₃), |
| | | 4.45 (t, 2H, NCH₂), |
| | | 7.27-7.36 (m, 1H, Ar—H), |
| | | 7.98 (b, 1H, NH) |
| | | Compound 40 |
| 511.14 | δ: | 0.94-0.98 (d, 6H, 2xCH₃), |
| | | 1.98 (m, 3H, Ar—CH₃), |
| | | 2.28 (s, 2H, CH₂), |
| | | 2.85 (t, 2H, NCH₂), |
| | | 3.42 (s, 3H, NCH₃). |
| | | 4.13 (t, 3H, CH₃), |
| | | 6.97-7.01 (m, 2H, 2xAr—H), |
| | | 7.69 (s, 1H, imidazole-H), |
| | | 1.4 (t, 2H, CH₂), |
| | | 2.24-238 (m, 1H, CH), |
| | | 2.70 (t, 4H, 2xCH₂), |
| | | 3.04 (t, 4H, 2xCH₂), |
| | | 3.93-3.96 (d, 2H, CH₂), |
| | | 4.45 (t, 2H, NCH₂), |
| | | 7.27-7.36 (m, 1H, Ar—H), |
| | | 7.98 (b, 1H, NH) |

TABLE 2
Rabbit Carvernosal Relaxation IC₅₀

| | PDE₅ | Rabbit Corpus Cavernosal |
|---|---|---|
| compound | (human platelet) IC₅₀(nM) | Relaxation IC₅₀ |
| 4 | 3.9 ± 0.1 | 7.16 ± 0.09 |
| 7 | 4.2 ± 0.2 | 7.13 ± 0.06 |
| 14 | 3.8 ± 0.2 | 7.84 ± 0.08 |
| 17 | 6.2 ± 0.1 | 7.64 ± 0.07 |
| 23 | 5.2 ± 0.1 | 7.38 ± 0.04 |
| 26 | 4.8 ± 0.2 | 7.42 ± 0.09 |
| 34 | 0.4 ± 0.2 | 8.13 ± 0.05 |
| 37 | 0.3 ± 0.1 | 8.03 ± 0.04 |
| 35 | 0.4 ± 0.1 | 8.25 ± 0.06 |
| 38 | 0.6 ± 0.2 | 8.16 ± 0.07 |
| 39 | 0.6 ± 0.2 | 8.16 ± 0.07 |
| 42 | 0.6 ± 0.2 | 8.16 ± 0.07 |
| 40 | 0.6 ± 0.1 | 8.27 ± 0.04 |
| 41 | 0.8 ± 0.2 | 8.30 ± 0.07 |

TABLE 3
Rabbit Corpus Carvernosal Relaxation IC₅₀ on K⁺ channels blocker

| Drug pretreatment | Dose | -Log EC₅₀ |
|---|---|---|
| Control | | 7.19 ± 0.09 |
| TEA | 10 mM | 5.037 ± 0.05 |
| Glibenclamide | 1 μM | 6.57 ± 0.15 |
| 4-AP | 100 μM | 5.83 ± 0.17 |
| L-NAME | 100 μM | 6.51 ± 0.08 |
| Methylene blue | 10 μM | 6.51 ± 0.06 |
| ODQ | 1 μM | 6.79 ± 0.12 |

TABLE 4
Peak increased intracarvernous pressure (ΔICP) and duration of
tumescense response to compounds 2 mg/kg in rabbits

| compound | ΔICP (mmHg) | Duration (min) |
|---|---|---|
| Compound 10 | 12 ± 1.6 | 13 ± 2.1 |
| Compound 4 | 14 ± 2.1 | 14 ± 1.1 |
| Compound 9 | 25 ± 1.3 | 16 ± 1.2 |
| Compound 12 | 23 ± 1.5 | 15 ± 1.3 |
| Compound 18 | 26 ± 1.4 | 18 ± 1.3 |

### Pharmaceutical Activity

The pharmaceutical activity of the compounds of this invention have been proven by the following pharmaceutical experiments.

Compound 14 was dissolved in 10% absolute alcohol, 10% propylene glycol and 2% 1 N HCl at 10 mM. Dilutions were made in distilled water. Glibenclamide was dissolved in 20% absolute alcohol and 80% DMSO (dimethyl sulfoxide). Other drugs were dissolved in normal saline.

In vitro Drugs

Compound 14 was dissolved in 10% absolute alcohol, 10% propylene glycol and 2% IN HCl at 10 Mm-10⁻² M. Dilutions were made in redistilled water. 10⁻³ M glibenclamide was dissolved in 95% absolute alcohol and 80% DMSO (dimethyl sulfoxide). Other drugs were dissolved in normal saline. 10⁻¹M dilutions were made in distilled water. 10⁻²M 1H-[1,2,4]Oxadiazolo[4,3-alquinoxalin-1-one(ODQ) (1H-[1,2,4]Oxadiazolo[4,3-a]quinoxalin-1-one, was dissolved in 100% DMSO (dimethyl sulfoxide), 10⁻³M dilutions were made in absolute alcohol, 10⁻⁴ M dilutions were made in redistilled water. IBMX was dissolved in 10% DMSO, 10⁻² dilutions was made in redistilled water. 10⁻²M Levcromakalim was dissolved in 50% DMSO, dilutions were made in 50% redistilled water. 8-Phenyltheophylline was dissolved in 80% absolute alcohol and 10⁻²M dilutions were made in 20% 0.2 M NaOH, while 10⁻³M dilutions were made in redistilled water. 10⁻²M XAC (Xanthineamine congener) was dissolved in redistilled water and 10⁻³M dilutions were made in redistilled water and 10⁻² M 8-(3-chlorostyryl) caffeine (CSC) was dissolved in DMS0, 10⁻³ M dilutions were made in redistilled water. 10⁻² M alloxazine was dissolved in 100% absolute alcohol, 10⁻³ M dilutions were made in redistilled water. Other drugs were dissolved in distilled water.

The Krebs solution (mM) comprises NaCl 118, KCl 4.8, CaCl₂ 2.5, MgSO₄ 1.2, KH₂PO₄ 1.2, NaHCO₃24, and glucose 11.

### Rabbit Corpus Carvernosal Strip Assay

Adult rabbit, weighing 200-300 g were abdominally anesthetized with sodium pentobarbital. The rabbit corpus carvernosal was removed immediately and placed in the Kreb solution equiliberated to a mixture of 95% O₂ and 5% CO₂ at room temperature (20-25° C.). After the surrounding tissue was carefully removed, the trachea was cut into spiral shape with every turn having 5 mm segments, the two ends of the corpus carvernosal were clamped with frog-heart shaped clamps, one end was fixed at the bottom of 20 ml of a tissue bath made of physiological saline solution. The temperature was maintained at 37° C. The other end of the corpus carvernosal was connected to a force transducer and isometric contractions and beating rate of the right atria was recorded by COULBOURN AT-High-Speed Video Figure. After the samples were given 200 mg of contractions and reached equilibrium the following experiments were carried out:

(a) cumulative concentration-response curves

After the equilibrium was reached again for at least 60 minutes, different concentrations of the drug were used. For evaluation of the activity of rabbit corpus carvernosal, cumulative administration of drug 1×10⁻⁹˜1×10⁻⁴ were carried out, and cumulative dose-response curve was obtained. The cumulative dose-response curve was the control group. The following experiments were performed:

(b) To examine the possible mechanisms of relaxation effects on corpus carvernosal, the aorta preparation of rats and rabbit was used.

(i) It was also observed whether the relaxation effects of corpus carvernosal are affected by K⁺ channels.

Some of the K⁺ channels blocker eg. 10 mM tetraethylammonium chloride (TEA), 1 μ, M glibenclamide and 100 μM 4-aminopyridine (4-AP) were used prior to the addition of the compound. Cumulative administrations of the drug 1×10⁻⁸˜1×10⁻⁴M were carried out again, and the inhibition of K⁺ channels blocker was obtained.

(ii) It was also observed whether the relaxation effects of corpus carvernosal are affected by cyclic GMP levels.

Pretreatment with 100 μM N^{w}-nitro-L-arginine methyl ester (L-NAME) 10 μM methylene blue and 1 μM ODQ was carried out. Then cumulative applications of drug 1×10⁻⁹˜1×10⁻⁴ M were carried out. Inhibitions by NOS inhibitor were obtained.

(iii) It was also observed whether the relaxation effects of corpus carvernosal are affected by PDE (phosphodiesterase) inhibition.

1 μM phenylephrine solution was added to induce vasoconstriction and when the vasoconstruction reached stability, the product was repeatedly washed with Kreb's solution and 0.5 μM phenylephrine was added to cause contraction, again.

When the contraction reached the maximum, 1 μM IBMX (3-Isobutyl-1-Methylxanthine) was administered first, then 0.01 μM˜0.1 μM compound 14 was added, to determine whether corpus carvernosal relaxation of compound 14 are affected by IBMX.

Result

The Mechanisms of Corpus Carvernosal Relaxation

10 μM phenylephrine solution were added to induce vasoconstrction and when the vasoconstriction reached stability, cumulative administration of the drug 1×10⁻⁹˜1×10⁻⁴ M were carried out. Keeping relaxation cumulative dose-response curve as control group, the following experiments were performed:

(i) FIG. 4 and Table 3 show the cumulative concentration-response curves to compound 14 against those K⁺ channels blocker, eg. TEA, glibenclamide and 4-AP pretreated the rabbit corpus carvernosal. This study shows-that compound 14 has corpus carvernosal relaxation activator with K⁺ channels opening and cGMP breakdown inhibition activities.

(ii) The PDE (phosphodiesterase) inhibition activity affected the corpus carvernosal relaxation activity of compound 14.

10 μM phenylephrine solution were added to induce vasoconstruction of endothelium-intact corpus carvernosal. When the vasoconstruction reached stability, the concentration-dependent vasorelaxations was produced by compound 14 (0.1, 0.5, and 1.00 μM). The relaxation cumulative dose-response curve was the control group. The endothelium-intact corpus carvernosal was repeatedly washed with Kreb's solution to move phenylephrine, until after 60 minutes it reached stability, then the following experiments were performed:

10 μM phenylephrine solution was added to the tissue bath to induce the vasoconstruction of endothelium-intact corpus carvernosal. When the vasoconstriction reached stability, the phenylephrine was repeatedly washed with Kreb's solution. Then 10/μM phenylephrine was administered. When the corpus carvernosal vasoconstriction reached stability IBMX (0.5 μM) was first administered, then compound 14 (01., 0.5, and 1.0 μM) was added. FIG. 5 shows the relaxation cumulative concentration-response curves and IBMX has an additive effect of compound 14.

Phosphodiesterase Five Assay

Phosphodiesterase 5 (PDE₅) activity was determined as described by Seiler, S. et al. in which, [³H]cGMP is used as the substrate of the human platelets homogenates to determine the PDE activity, using Scintillation Proximity assay kit(SPA). Table 2 shows the inhibitory effect of phosphodiesterase in platelet.

Measurement of ICP

Male New Zealand white rabbits weighing 2-3 kg were used for measurement. After sedation with an intramuscular injection of ketamine 10 mg, the rabbits were anesthetized with intraperitoneal sodium 30/kg as needed. The animal breathed spontaneously. The rabbits were then placed in the supine position, and the body temperature was maintained at 37° C. using a heating pad and lamp. The femoral artery on one side was cannulated for monitoring of continuous systemic arterial pressure, the mean systemic arterial pressure and the heart rate via a Gould 23 ID pressure transducer were determined. Under sterile conditions, the skin overlying the penis was incised and the corpora carvernosa were exposed at the root of the penis. A 25-gauge needle was inserted into the corpus carvernosum for pressure recording (Gould, RS-3400). The needle was connected to a three-way stopcock, thus permitting the intracarvernous injection of drugs. The tube was filled with heparizized saline (50 IU/2-3 h) to prevent clotting. Table 4 shows the increase of intracarvernous pressure induced by the compounds of the invention.

### Example 1

### Synthesis of Compound D

0.2 mole 3-isobutyl-1-methylxanthine (IBMX) was dissolved in 0.4 mole 2-bromoethylamine solution, and the solution was stirred at 100° C. mantle heater until the solid completely melted. The 125 ml 1.6 N NaOH was added and the reaction was carried out for 3-5 hours under 150° C. to complete the reaction. Then the product was concentrated under reduced pressure to obtain white coarse crystals which were recrystallized from methanol to obtain the pure white crystal compound D (N7-2aminoethyl 3-isobutyl-1-methylxanthine).

### EXAMPLE 2

### Synthesis of Compounds 39-41

One mole of para-hydroxyl sulfonic acid was reacted with 1 mole of chlorosulfonic acid for 30 minutes, then the liquid was poured into ice water. The precipitate of para-hydroxy sulfonyl chloride was collected and dried under reduced pressure.

With proper amount of methanol to dissolve the precipitate, 1 mole of methylpiperazine was added and reacted for 2 hrs. The product was dissolved in 4 mole 30% formalin, then the same mole amount of compund D(N7-N-7-2 aminoethyl 3-isobutyl-1-methyl-xanthine) was added. Then 1% acetic acid-methanol solution was added to obtain compound 39 after 24 hours reaction. The product was purified through a silica gel column and was dissolved in methanol, 4% NaOH was added and 1 mole ethyl bromide was added to obtain compound 40. By replacing ethyl bromide with propyl bromide compound 41 was obtained.

### EXAMPLE 4

### Synthesis of Compounds 33-35

Para-hydroxybenzoic acid was reacted with ethanol to obtain para-hydrobenzoic acid ethyl ester by mediated with SOCl₂. The product was added to 33% NH_{3(aq)},refluxed for 1 hour and recrystallized from methanol to obtain para-hydroxybenzoic amide. Formalin 4 moles times of para-hydroxybenzoic-amine solution was added in methanol, then the Mannich reaction was carried out with N7-bromoethylaminate 3-isobutyl-1-methylxanthine and trace of acetic acid. The product of compound 33 was obtained which was purified by silica gel chromatography.

The column was eluted with ethyl alcohol and methanol, the solution was concentrated under reduced pressure, recrystallized from methanol to obtain compound 33. Compound 33(1 mole) was dissolved in 100 ml methanol, reacted with 4% NaOH and 1 mole ethyl bromide, to obtain compound 34. Accordingly, compound 35 was obtain by replacing ethyl bromide with propyl bromide.

### EXAMPLE 5

### Synthesis of Compounds 36-38

Following the process described in Example 4 and replacing theophylline with IBMX, separately, compounds 36, 37 and 38 were obtained.

### EXAMPLE 6

### Synthesis of Compound A

0.2 mole of 3-isobutyl-1-methylxanthine (IBMX) was dissolved in methanol and mixed with 0.4 mole 1,2-dibromoethane solution. Then the solution was stirred at 100° C. in a mantle heater until the solid was completely melted. Then 125 ml 1.6N NaOH was added to react for 3-5 hours at 150° C. The reaction mixture was concentrated under reduced pressure to obtain white coarse crystals, which were recrystallized from methanol to obtain the white crystal compound A (N7-bromoethyl 3-isobutyl-1-methylxanthine).

### EXAMPLE 7

### Synthesis of Compound 21

Two moles benzenesulfonyl chloride and 2 moles piperazine were dissolved in methanol and refluxed for 1 hour. The solution was concentrated under reduced pressure, recrystallized from methanol to obtain benzenesulfonyl piperazine. One mole of the product was dissolved in methanol, 1 mole of compound A (N7-bromoethyl 3-isobutyl-1-methylxanthine) was added and refluxed for 8 hours. The solution was concentrated under reduced pressure, purified by a silica gel column chromatography, eluted with methanol and ethyl acetate, concentrated under reduced pressure, recrystallized from methanol to obtain compound 21.

### EXAMPLE 8

### Synthesis of Compounds 22-23

Following the process of Example 7 and replacing p-toluene-sulfonyl chloride or o-toluenesulfonyl chloride with benzenesulfonyl chloride compounds 22 and 23 were obtained.

### EXAMPLE 9

### Synthesis of Compounds 24-26

Following the process of Example 6 and 7 and replacing theophylline with IBMX compounds 24, 25 and 26 were obtained.

### EXAMPLE 10

### Synthesis of Compounds 9 and 10

One mole theophylline was dissolved in methanol and 3 moles of 1,2-dibromoethane were added with 2 mole NaOH to neutralization and the mixture was refluxed 5 hours. It was then concentrated under reduced pressure and purified by chromatography, eluted with methanol and ethyl acetate, then concentrated under reduced pressure to obtain compound A. Compound A was dissolved in methanol, 0.8 mole piperazine was added the mixture refluxed and then concentrated under reduced pressure. The product B was obtained.

Compound B was dissolved in methanol, then 2-Furoyl Chloride or 4-chloronitrobenzene were added, to reflux, then concentrated under reduced pressure, and the product was recrysallized from methanol to obtain compound 9 or 10.

### EXAMPLE 11

### Synthesis of Compounds 1-8

Compound A was dissolved in methanol. Then piperazine, 1-Phenylpiperazine, 1-(2-Pyrimidyl)piperazine, 1-(2-Pyridyl)piperazine, N-Benzylpiperazine, 1-(2-Chlorophenyl)piperazine, 1-(o-Methoxyphenyl)piperazine, 1-(m-Chlorophenyl)piperazine, or 1-(4-Chlorophenyl)piperazine was added, to reflux reaction to obtain compounds 1-8.

### EXAMPLE 12

### Synthesis of Compounds 11˜18

By following the process of Example 11 and replacing theophylline with IBMX, compounds 11˜18 where obtained.

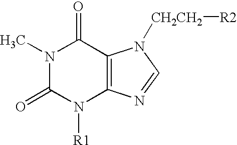

| Compound | | | | | |
|---|---|---|---|---|---|
| No. | R₁ | R₂ | R₃ | R₄ | R₅ |
| 27 | CH₃ | 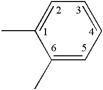 | — | H | OH on 6 |
| 28 | CH₃ | 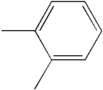 | — | H | C₂H₅O on 6 |
| 29 | CH₃ | 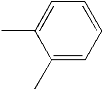 | — | H | C₃H₇O on 6 |
| 30 | CH₃ | 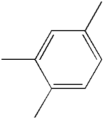 | — | CONH₂ on 3 | HO on 6 |
| 31 | CH₃ | 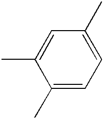 | — | CONH₂ on 3 | C₂H₅O on 6 |
| 32 | CH₃ | 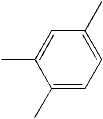 | — | CONH₂ on 3 | C₃H₇O on 6 |
| 33 | CH₃ | 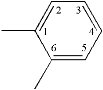 | — | HO on 6, CH₃ on 4′, | 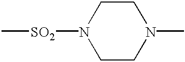 |
| 34 | CH₃ | 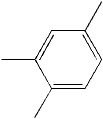 | — | C₂H₅O on 6, CH₃, on 4′, | 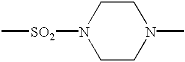 |
| 35 | CH₃ | 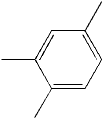 | — | C₃H₇O on 6 CH₃ on 4′, | 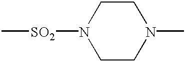 |
| 36 | —CH₂CH(CH₃)₂ | 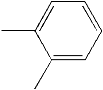 | — | H | OH on 6 |
| 37 | —CH₂CH(CH₃)₂ | 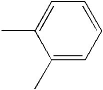 | — | H | C₂H₅O on 6 |
| 38 | —CH₂CH(CH₃)₂ | 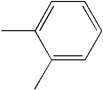 | — | H | C₃H₇O on 6 |
| 39 | —CH₂CH(CH₃)₂ | 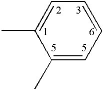 | — | CONH₂ on 3 | HO on 6 |
| 40 | —CH₂CH(CH₃)₂ | 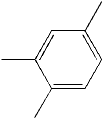 | — | CONH₂ on 3 | C₂H₅O on 6 |
| 41 | —CH₂CH(CH₃)₂ | 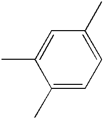 | — | CONH₂ on 3 | C₃H₇O on 6 |

| | |
|---|---|
| | [II] |
| 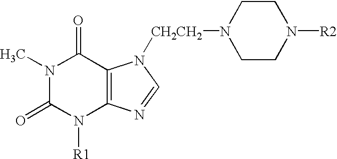 | |

| Compound | | | | | |
|---|---|---|---|---|---|
| No. | R1 | R2 | R3 | R4 | R5 |
| 1 | CH₃ | 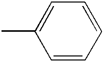 | — | H | H |
| 2 | CH₃ | 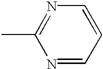 | — | H | H |
| 3 | CH₃ | 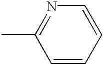 | — | H | H |
| 4 | CH₃ | 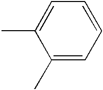 | — | Cl on 2 | H |
| 5 | CH₃ | 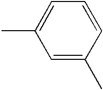 | — | Cl on 4 | H |
| 6 | CH₃ | 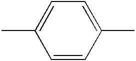 | — | Cl on 3 | H |
| 7 | CH₃ | 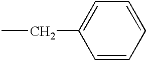 | — | H | H |
| 8 | CH₃ | 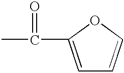 | — | H | H |
| 9 | CH₃ | 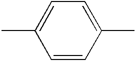 | — | No on 3 | H |
| 10 | CH₃ | 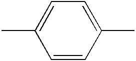 | — | CH₃O on 3 | H |
| 11 | —CH₂CH(CH₃)₂ | 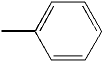 | — | H | H |
| 12 | —CH₂CH(CH₃)₂ | 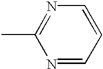 | — | H | H |
| 13 | —CH₂CH(CH₃)₂ | 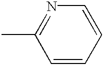 | — | H | H |
| 14 | —CH₂CH(CH₃)₂ | 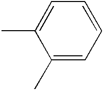 | — | Cl on 2 | H |
| 15 | —CH₂CH(CH₃)₂ | 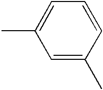 | — | Cl on 4 | H |
| 16 | —CH₂CH(CH₃)₂ | 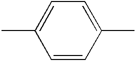 | — | Cl on 3 | H |
| 17 | —CH₂CH(CH₃)₂ | 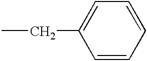 | — | H | H |
| 18 | —CH₂CH(CH₃)₂ | 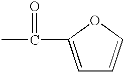 | — | H | H |
| 19 | —CH₂CH(CH₃)₂ | 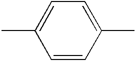 | — | NO₂ on 3 | H |
| 20 | —CH₂CH(CH₃)₂ | 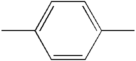 | — | CH₃O on 3 | H |
| 21 | CH₃ | 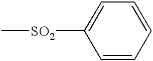 | — | — | — |
| 22 | CH₃ | 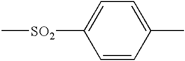 | — | CH₃O on 3 | — |
| 23 | CH₃ | 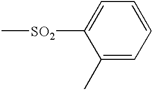 | — | CH₃O on 6 | — |
| 24 | —CH₂CH(CH₃)₂ | 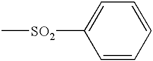 | — | — | — |
| 25 | —CH₂CH(CH₃)₂ | 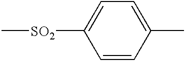 | — | CH₃O on 3 | — |
| 26 | —CH₂CH(CH₃)₂ | 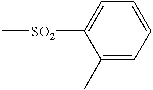 | — | CH₃O on 6 | — |

The names of compounds 4 and 14 are as follows:

Compound 4:

N7-{2-[4(2-chlorophenyl)piperazinyl]ethyl},1,3-dimethyl xanthine

Compound 14:

N7-{2-[4(2-chlorophenyl)piperazinyl]ethyl},3-isobutyl-1-methyl xanthine

What is claimed is:

1. A compound containing the theophylline moiety of formula I, 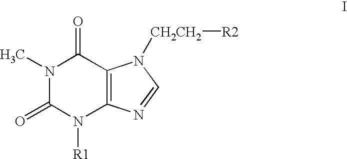
wherein
R1 is —(CH₂)₂CH₃;R₂ is a member selected from the group 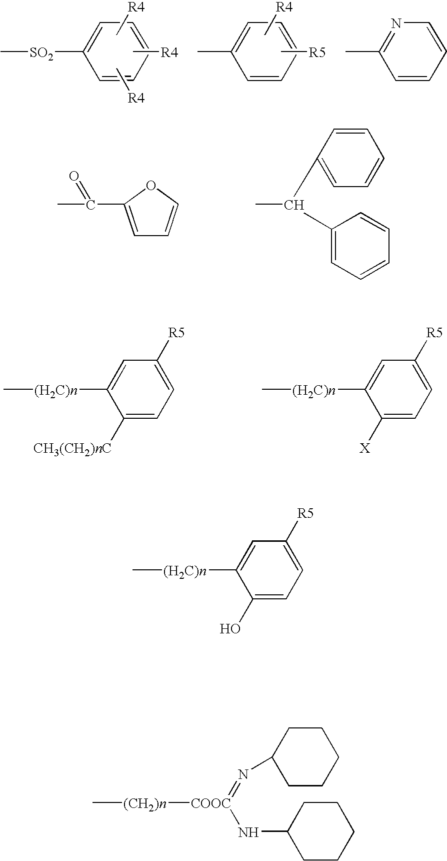
wherein R4 is a member selected from the group of H, —(CH₂)ₙ CH₃, X,—NH₂ and —NO₂ and X designates F, Cl, Br or I
wherein R₅ is a member selected from the group of H, 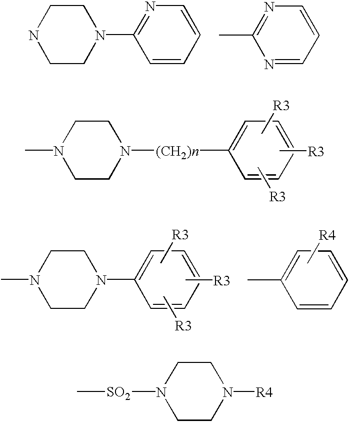
wherein
R3 is a member selected from the group of halogen, hydroxyl group, a saturated straight chain of 1-3 carbon atoms and when one of R3 is hydrogen, n is 0, 1, 2 or 3.

2. A compound containing the theophylline moiety of formula II 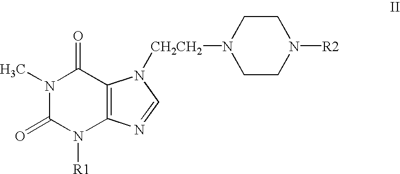
wherein
R1 is —(CH₂)ₙ; CH₃; R2 is a member selected from the group of 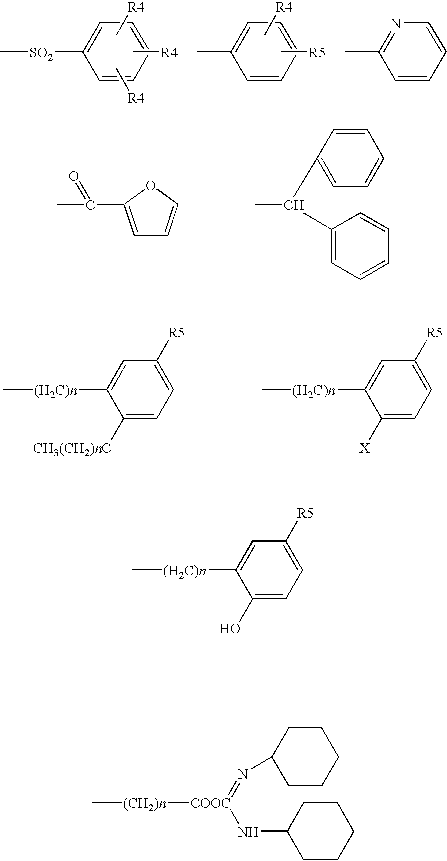
wherein
R4 is a member selected from the group of H, —(CH₂)ₙ CH₃, X, —NH₂ and —NO₂; X designates F, Cl, Br or I.
R3 is a member selected from the group of H, 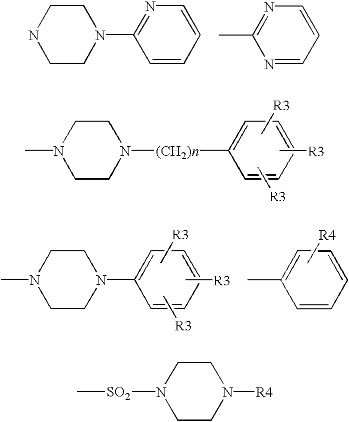
R3 is a member selected from the group of halogen, hydroxyl group (OH), a saturated straight chain of 1-3 carbon atoms and when one of R3 is hydrogen; n is 0, 1, 2 or 3.

3. A pharmaceutical composition which has corpus carvernosal relaxation activity containing a compound of formula I as defined in claim 1.

4. A pharmaceutical composition which has corpus carvernosal relaxation activity containing a compound of formula II as defined in claim 2.

5. A process for the preparation of a compound of formula I according to claim 1 which comprises the reacting of a compound of formula 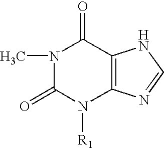
in which R₁=CH₂CH(CH₃)₂ or R₁=CH₃
with 1,2-dibromoethane as shown in step a 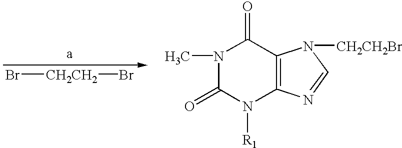
to produce a monobromo compound which is then reacted in step b with the piperazinyl ring which is a secondary amine, then adding NaOH to precipitate NaBr and obtaining the product which contains the piperazinyl ring.

6. A process for the preparation of a compound of formula II according to claim 2 which comprises the reaction of a compound of formula 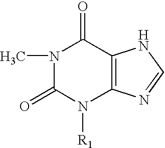
R₁=CH₂CH(CH₃)₂ or R₁=CH₃
with 1,2-dibromoethane
to produce a monobromo compound according to reaction a 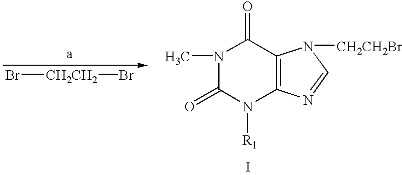
and said monobromo compound is reacted with a N-substituted piperazine according to reaction b or with piperazine according to reaction c to produce a compound wherein the N is not substituted 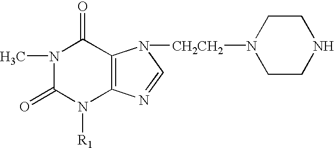
and said compound is reacted according to d to produce a compound II [chemical structure]
